(19) United States
(12) Patent Application Publication
(10) Pub. No.: US 2011/0208310 A1
(43) Pub. Date: Aug. 25, 2011

(54) ULTRASONIC WELDABLE SPINAL IMPLANTS AND RELATED METHODS

(76) Inventors: Felix Aschmann, Oberdorf (CH); Heinz Michael Mayer, Graefelfing (DE)

(21) Appl. No.: 13/012,344

(22) Filed: Jan. 24, 2011

Related U.S. Application Data
(60) Provisional application No. 61/306,012, filed on Feb. 19, 2010.

(51) Int. Cl. A61F 2/44

(52) U.S. Cl. 623/17.16

(57) ABSTRACT

A spinal implant for augmenting or supporting a patient's spine including vertebrae and intervertebral discs includes a first component constructed of a thermoplastic material and a second component constructed of the thermoplastic material. The first and second components are configured for implantation into the spine such that the first component contacts the second component at a welding point in an initial implanted position. An ultrasonic probe includes a tip that is configured to selectively contact the first component and/or the second component in the initial implanted position to transform the welding point to a welding joint in a final implanted position. The first component is fixed to the second component at the weld joint in the final implanted position.

### CROSS-REFERENCE TO RELATED APPLICATIONS

This application claims the benefit of U.S. Provisional Patent Application Ser. No. 61/306,012 filed Feb. 19, 2010, the disclosure of which is hereby incorporated by reference as if set forth in its entirety herein.

### BACKGROUND OF THE INVENTION

Ultrasonic welding techniques are utilized to assemble and secure thermoplastic materials together in several industries. However, the commercial utilization of ultrasonic welding has not been employed in orthopedic surgery and, specifically, in spinal surgery. Accordingly, it would be desirable to design and develop implants and methods for spine surgery that maximize the advantages of the ultrasonic welding techniques and adapt those techniques to the unique environment of spinal surgery.

### SUMMARY

Briefly stated, preferred embodiments of the present invention are directed to a spinal implant for augmenting or supporting a patient's spine including vertebrae and intervertebral discs. A spinal implant includes a first component constructed of a thermoplastic material and a second component constructed of the thermoplastic material. The first and second components are configured for implantation into the spine such that the first component contacts the second component at a welding point in an initial implanted position. An ultrasonic probe includes a tip that is configured to selectively contact the first and/or second component in the initial implanted position to transform the welding point to a weld joint in a final implanted position. The first component is fixed to the second component at the welded joint in the final implanted position.

### BRIEF DESCRIPTION OF THE SEVERAL VIEWS OF THE DRAWINGS

The foregoing summary, as well as the following detailed description of preferred embodiments of the instruments, implants and methods of the present application, will be better understood when read in conjunction with the appended drawings. For the purposes of illustrating the ultrasonic weldable spinal implants, instruments and methods of the present application, there are shown in the drawings preferred embodiments. It should be understood, however, that the application is not limited to the precise arrangements and instrumentalities shown. In the drawings:

FIGS. 1A-1C illustrate side elevational, top plan and front elevational views of several augmentation implants in accordance with a first preferred embodiment of the present invention implanted in a vertebra;

FIGS. 2A-2C illustrate top plan and side elevational views of an ultrasonic weldable implant for modular cages in accordance with a second preferred embodiment of the present invention mounted within or being mounted within an intervertebral disc space of a patient's spine;

FIGS. 3A and 3B illustrate side elevational and top plan views of an ultrasonic weldable vertebral body replacement device in accordance with a third preferred embodiment of the present invention, mounted between two vertebrae of a patient's spine;

FIGS. 4A and 4B illustrate rear elevational and side elevational views of an ultrasonic weldable annulus repair implant in accordance with a fourth preferred embodiment of the present invention, mounted to the patient's spine;

FIG. 5 illustrates an ultrasonic weldable cage ancoring implant in accordance with a fifth preferred embodiment of the present invention, mounted to the patient's spine;

FIGS. 6A and 6B disclose ultrasonically weldable laminoplasty implants in accordance with a sixth preferred embodiment of the present invention, mounted to posterior portions of a patient's spine;

FIGS. 7A-7D illustrate an ultrasonically weldable interspinous plate in accordance with a seventh preferred embodiment of the present invention mounted between adjacent spinous processes of the patient's spine;

FIGS. 8A-8C illustrate several side elevational views of an ultrasonically weldable anterior interbody cage in accordance with an eighth preferred embodiment of the present invention, mounted in an intervertebral disc space of the patient's spine;

FIGS. 9A-9D illustrate side elevational views of an ultrasonically weldable rod in accordance with a ninth preferred embodiment of the present invention, mounted or being mounted between pedicle screws in the patient's spine;

FIG. 10 illustrates an ultrasonically weldable transverse connector in accordance with a tenth preferred embodiment of the present invention, mounted between adjacent rods in the patient's spine;

FIGS. 11A-11D illustrate side elevational, front elevational and top plan views of an ultrasonically weldable pedicle screw ancoring implant in accordance with an eleventh preferred embodiment of the present invention, mounted to a vertebra of the patient's spine;

FIGS. 12A and 12F illustrate an ultrasonically weldable, expandable innerspinous spacer in accordance with a twelfth preferred embodiment of the present invention; and

FIGS. 13A and 13B illustrate side elevational views of an ultrasonically weldable interspinous process blocking implant in accordance with a thirteenth preferred embodiment of the present invention, being mounted or mounted to the patient's spine.

### DETAILED DESCRIPTION

Certain terminology is used in the following description for convenience only and is not limiting. The words “right”, “left”, “lower” and “upper” designate directions in the drawings to which reference is made. The words “inwardly” or “distally” and “outwardly” or “proximally” refer to directions toward and away from, respectively, the patient's body, or the geometric center of the preferred ultrasonic weldable spinal implants and related parts thereof. The words, “anterior”, “posterior”, “superior,” “inferior”, “lateral” and related words and/or phrases designate preferred positions, directions and/or orientations in the human body to which reference is made and are not meant to be limiting. The terminology includes the above-listed words, derivatives thereof and words of similar import.

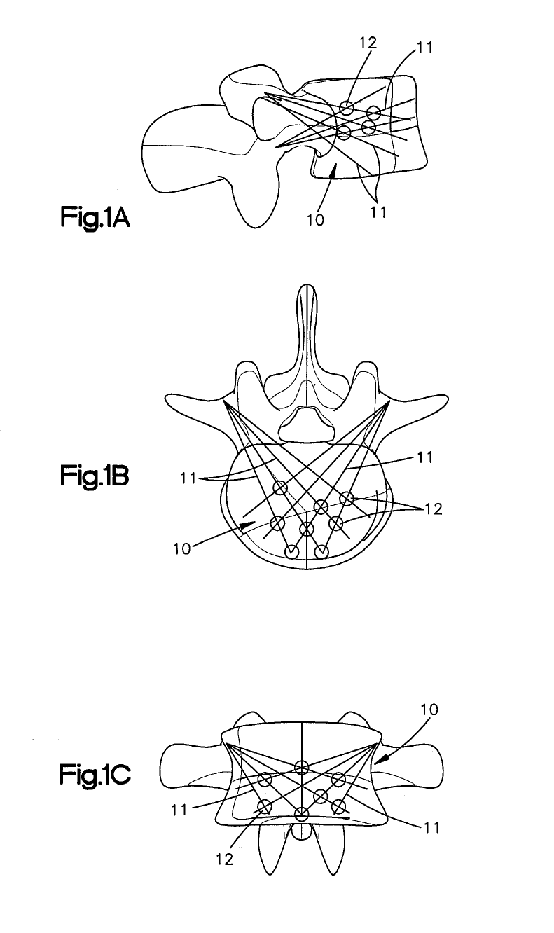

Referring to FIGS. 1A-1C, in treatment of an ostepenic spine (osteoporosis, cancer, etc.) implant anchorage, especially in the vertebral body, can be difficult due to the relatively soft composition of the impacted bone. Techniques such as vertebroplasty and kyphoplasty are sometimes used to improve implant anchorage and strength of the vertebral body by use of cement injected into the vertebral body. These techniques lack control of the position and shape of the cement and the materials and injection process are relatively difficult to handle.

In a first preferred embodiment, an augmentation ultrasonically weldable implant 10 is comprised of a three-dimensional intraosseous trellis-work implanted into the vertebral body including a multitude of thermoplastic pins 11 introduced through both pedicles into the vertebral body and welding these pins 11 to each other at welding points 12 at several areas in the vertebral body where the pins 11 contact each other. The pins 11 form a bundle within the pedicle and can be connected to any type of fixation element such as the shaft of a pedicle screw. The pins 11 are not limited to being introduced through the pedicles and may be introduced into the vertebral body through sidewalls and/or endplates of the vertebral bodies. In addition, the pins 11 are not limited to being inserted into the vertebral bodies and may be introduced into the lamina, disc space, interspinous processes, lateral mass or other portions of the patients spine or vertebrae to provide support, augmentation or fixation in an impacted area.

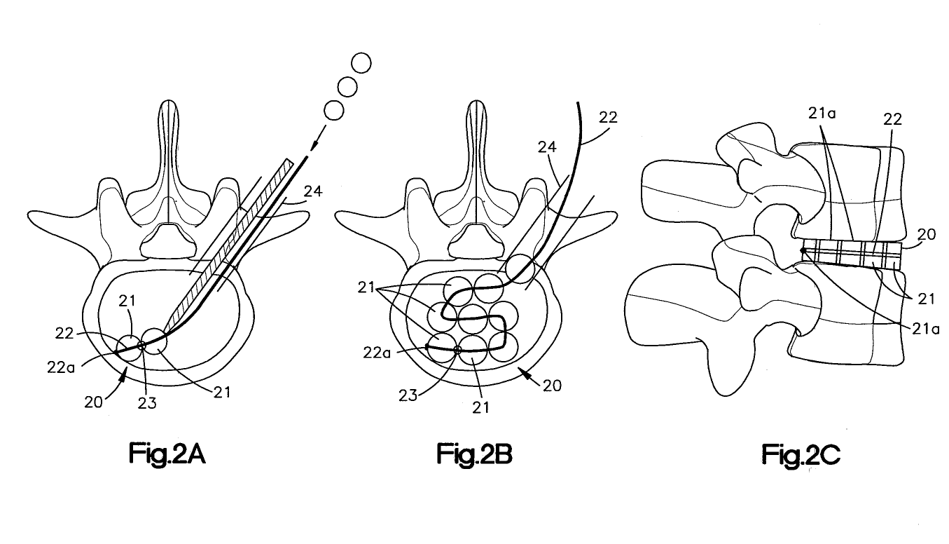

Referring to FIGS. 2A-2C, spinal fusion is generally performed by clearing the interbody space, placing an interbody spacer (cage) into the space and fixing the vertebral segment with pedicle screws and a rod (posterior approach). For the cage, it is desirable to offer wide contact surfaces to the vertebral bodies to achieve good stability and to prevent the cage from subsiding into the endplates of the vertebral bodies. On the other hand, the cage should be compact to allow implantation through a small incision to limit damage to a patient's soft tissue during surgery. Furthermore, the cage should adapt well to the endplate of the vertebral bodies to allow for stability and even load distribution.

In a second preferred embodiment, a modular ultrasonically weldable cage 20 may be assembled, in situ, in the interbody space of two adjacent vertebrae. Assembly can be achieved by building up the cage 20 from a plurality of cylinder-shaped pieces 21 that are introduced into the space along a wire or band 22 and are subsequently welded to each other in a final size and shape within the intervertebral space in an implanted position. The cylinder-shaped pieces 21 preferably have a bore to allow for bony in-growth or filling with bone graft. During insertion the cylinder-shaped pieces 21 can be controlled by sliding them on/along the wire or band 22 (preferably made of metal) into the interbody space, wherein the first cylinder-shaped pieces 21 is preferably held on the wire/band 22 by a stop 22a. The cylinder-shaped pieces 21 are preferably welded together piece by piece during insertion at weld spots or points 23 or all at once at the end of insertion of all of the cylinder-shaped pieces 21. Alternatively the cylinder-shaped pieces 21 can be controlled by a structure enclosing all of cylinder-shaped pieces 21, like a tie wrap or a pouch (not shown). The cylinder-shaped pieces 21 are preferably introduced into the disc space through a cannula 24 that may be manipulated to place the cylinder-shaped pieces 21 in preferred locations within the space. The cylinder-shaped pieces 21 preferably have generally planar upper and lower surfaces 21a that interact with the endplates of the vertebral bodies in the implanted position to provide stability to the final cage 20. The cylinder-shaped pieces 21 are preferably constructed of a thermoplastic material that accommodates welding of the cylinder-shaped pieces 21 at the welding spots 23 to form the final cage 20. The combination of the cannula 24, cylinder-shaped pieces 21 and wire 22 permit insertion of the final cage 20 through a relatively small incision and placement of the cylinder-shaped pieces 21 at user preferred positions, while also permitting formation of a relatively strong, solid final cage 20 to promote fusion between the vertebrae.

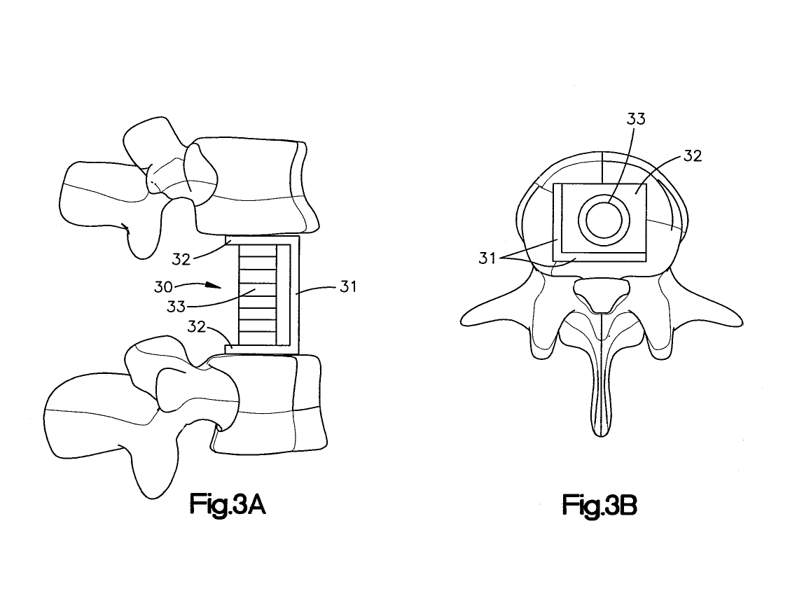

Referring to FIGS. 3A and 3B, in vertebral body replacement, it is desirable to use expandable implants because they allow for insertion through a relatively small access or incision and for adaptation to the needed height without the need of measuring or using trials. However, it is difficult to combine this technique with the use of bone graft and cement because there is typically no container for graft or cement containment.

In a third preferred embodiment, a modular ultrasonically weldable cage 30 provides a support for graft or cement. This support may be a “wall” 31 that can be welded to endplates 32 of the cage 30 in order to provide a barrier in any desired direction that prevents unwanted contact between the graft or cement and surrounding tissues. This wall 31 can be made of PEEK profiles that can be adapted and combined to create a barrier of the desired shape. The barrier or wall 31 may also be perforated to allow for infusion with blood. The endplates 32, wall 31 and expandable column 33 are preferably constructed of a thermoplastic material that permits movement of various pieces relative to each other during insertion and placement and accommodates fixation of the pieces together in a final implanted, generally expanded, position to secure a superior vertebra in position relative to an inferior vertebra.

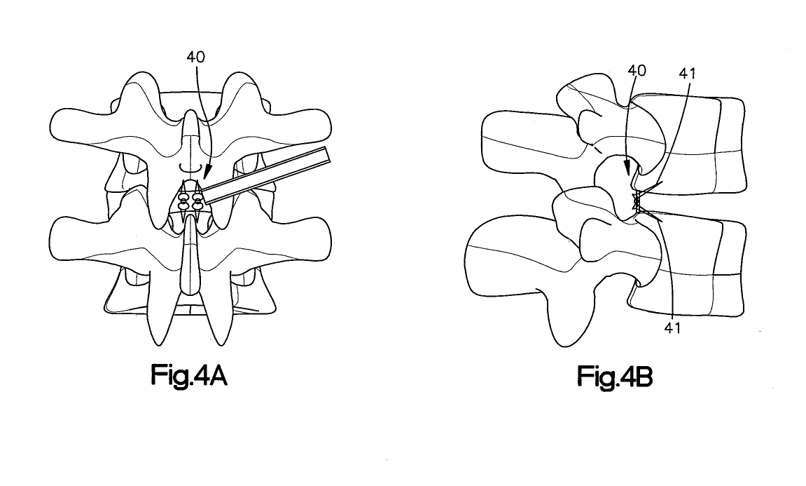

Referring to FIGS. 4A and 4B, in the process of disc degeneration, the annulus is often damaged and nucleus material extrudes through an annular defect. The extruded material often interferes with neural structures causing pain and dysfunction. A possible treatment can be the removal of the sequester (extruded nucleus material), but it is very probable that more nucleus material is extruded after such a surgical intervention. Therefore, it is desirable to close the annular defect after the sequester has been removed.

In a fourth preferred embodiment, an ultrasonically weldable annular repair implant 40 covers the annular defect with a mesh or membrane, wherein the mesh/membrane is attached to anchors 41 that previously are placed in the vertebral body. The attachment of the implant 40 to the anchors 41 is performed by ultrasonic welding. Alternatively the mesh/membrane or implant 40 can be placed first and the anchors 41 are inserted through the mesh/membrane 40 into the cortex of the vertebral bodies using ultrasonic energy and welding. The mesh/membrane and the anchors 41 are preferably constructed of a thermoplastic material that permits the ultrasonic welding of the components to each other in an implanted position.

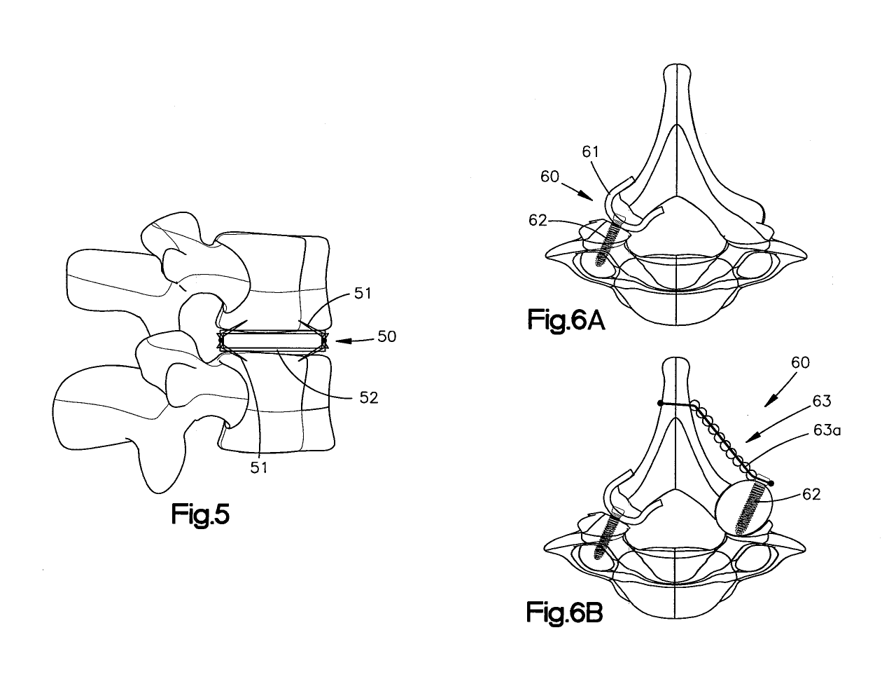

Referring to FIG. 5, in spinal interbody fusion, it is desirable to place a cage between the two vertebral bodies to fuse and to stabilize the spinal segment at the same time with screws that are inserted through the cage into the vertebral bodies. However, there are limitations to this technique, since the screws typically have to be placed in predefined positions and with predefined orientation. Furthermore, screw insertion is generally a lengthy process.

In a fifth preferred embodiment, an ultrasonically weldable cage implant 50 is comprised of screws or pins 51 inserted into the vertebral bodies through a cage 52. The pins 51 are preferably constructed of a thermoplastic material and the cage is preferably constructed of a thermoplastic material or includes an interface constructed of a thermoplastic material through which the pins 51 are inserted. The thermoplastic material is a polyether ether ketone (“PEEK”) material in the preferred embodiment, but is not so limited and may be constructed of another thermoplastic material or nearly any material that is capable of being ultrasonically weldable and is biocompatible. The screw/pin 51 preferably “drills” (weld/melt) a hole into the cage 52 (or PEEK interface) at the position where the surgeon wants to place the screw/pin 51. Thus the screw/pin 51 can be placed at any position and with any orientation desired relative to the cage 52 and/or the vertebra. At the same time the screw/pin 51 is welded to the cage 52 or the PEEK interface. Alternatively the cage 52 or PEEK interface therein can have holes to accommodate the screw/pin 51, wherein the hole is shaped in a manner that allows the screw/pin 51 to be oriented in a wide range of angles (similar to polyaxial pedicle screw heads).

Referring to FIGS. 6A and 6B, in cervical spine surgery, a common way for treating stenosis is to perform laminoplasty with a Hinge/Open-door technique. In this type of treatment, a plate is used in combination with screws to keep the semi-dissected lamina in the open-door position. This technique typically requires placement of four screws to allow for stable fixation of the lamina.

In a sixth preferred embodiment, an ultrasonically weldable laminoplasty implant 60 includes a U-shaped spacer 61 where the U-shape accommodates the lamina at the site of dissection and a screw or pin 62 that is designed to be inserted into the pedicle at the site of the dissection (masa lateralis). The spacer 61 and the screw/pin 62 can be comfortably placed separately and then the spacer 61 is welded to the screw/pin head by means of ultrasonic power. Accordingly, the spacer 61 and screw/pin 62 are constructed of a thermoplastic material or other material that is appropriate for ultrasonic welding purposes. Alternatively the screw/pin 62 can be placed on the opposite side of the dissection and the lamina is stabilized by a string 63 attached to the spinous process on one end and to the screw/pin head 62 on the other end. The string 63 has a plurality of PEEK segments 63a (bead-like) attached to it along its entire length. The length of the string 63 is adapted intra-operatively to the desired length and the PEEK segments 63a are welded one to another to transform the string 63 into a rod. Thus the lamina is completely stabilized since the (now) rod can take loads in tension, compression, shear, bending and torsion.

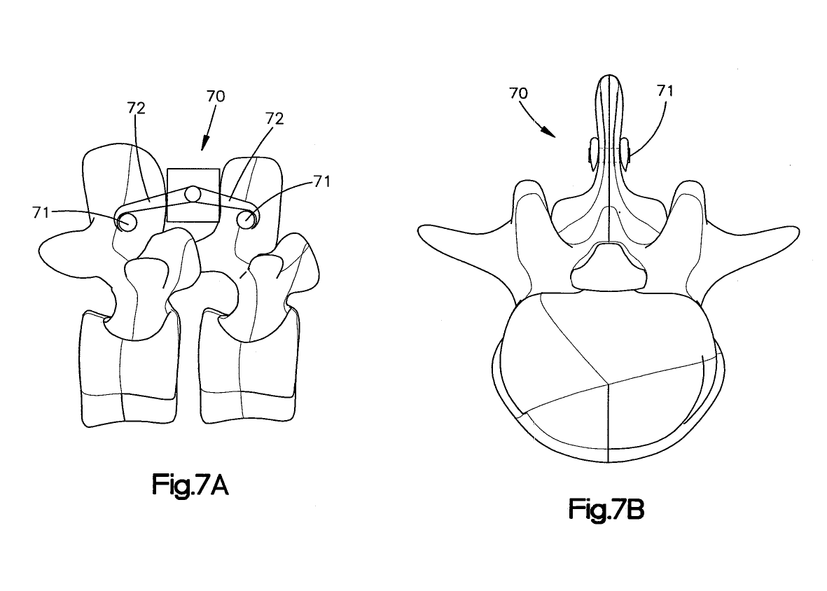

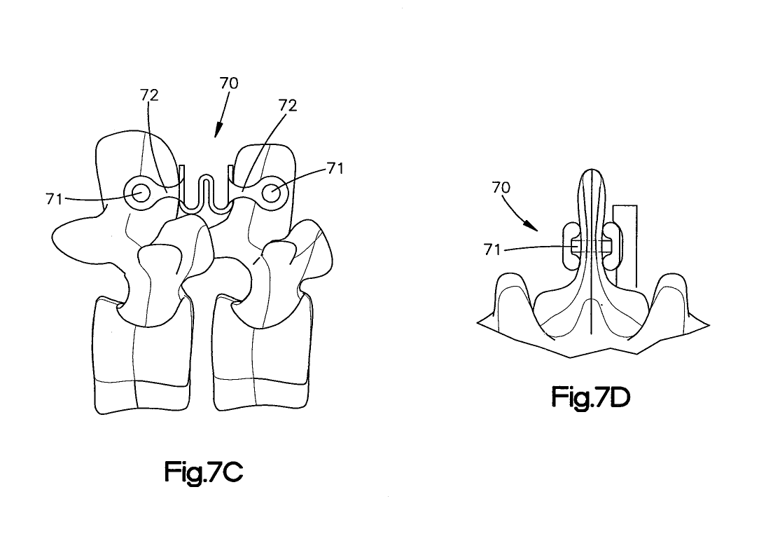

Referring to FIGS. 7A-7D, in lumbar spinal fusion, a technique is to place an interbody cage between the vertebral bodies to fuse and to stabilize the segment with pedicle screws and rods. However, placing pedicle screws is a relatively invasive and time consuming procedure. It is desired to have a less invasive and faster procedure for fixation of vertebral bodies in a lumbar spinal fusion.

In a seventh preferred embodiment, an ultrasonically weldable interspinous process spacer 70 attaches to the spinous processes of the segment to fuse. The spacer 70 preferably, mainly reacts compressive loads and, therefore, lock extension and the portion of the implant attaching to the spinous processes will stabilize all other motion of the segment to fuse. Pins/bolts 71 may be placed into the spinous processes serving as anchorage for the spacer 70 to be inserted subsequently. The spacer 70 preferably has projections 72 that extend cranially and caudally on either side of the spinous processes and can be attached to the anchorage or pins/bolts 71 by means of ultrasonic welding. Alternatively the spacer 70 may be placed first and the anchorage 71 subsequently. In this case the ancorage 71 will “drill” holes through the spinous processes and weld to the projections of the spacer 70 by of ultrasonic power. The devices above described may be implanted through a standard posterior approach as well as a minimally invasive, percutaneous lateral approach.

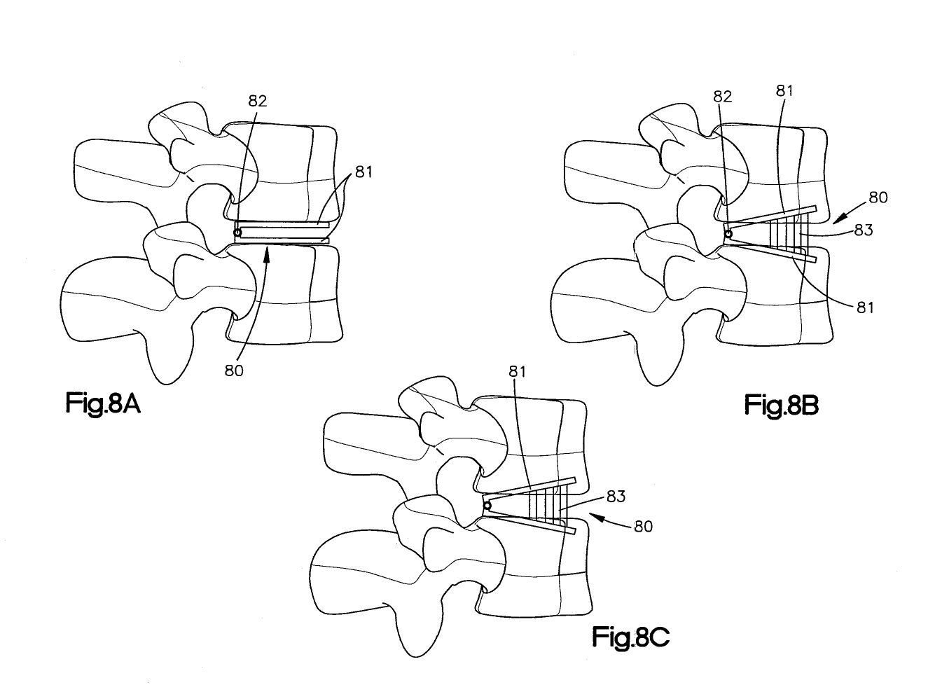

Referring to FIGS. 8A-8C, in spinal fusion, it is a common problem to achieve desired distraction and lordotic angle since common cages don't allow for adaptation of either angle or height. In general, the surgeon has a variety of cages of different angulation, shape and height. The best fit is determined by positioning trials corresponding to the cage geometry between the adjacent vertebral bodies.

In an eighth preferred embodiment, an ultrasonically weldable cage 80 allows for in situ adjustment of height and lordotic angle. The interbody spacer 80 is comprised of two endplates 81 connected to each other by a hinge 82. When inserted between the vertebral bodies the spacer 80 is in a folded configuration. Once in a surgeon preferred position the spacer 80 can be distracted to the desired angle and fixed at the desired angle by inserting a wedge-shaped spacer 83 between the endplates 81 and welding the wedge-shaped spacer 83 to the endplates 81. Alternatively the two endplates 81 of the interbody spacer 80 can be connected by a mechanism allowing telescoping and angular movement between each other. Therefore, adjustment of an angle and a height can be performed independently. Locking of the endplates 81 to each other is obtained by welding the PEEK pin or wedge 83 to the endplates 81 or by overlapping regions of the endplates 81 to each other by ultrasonic power proximate the hinge 82.

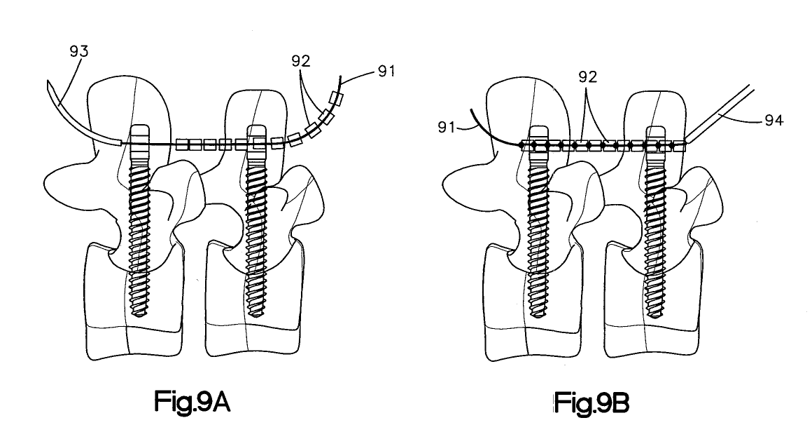

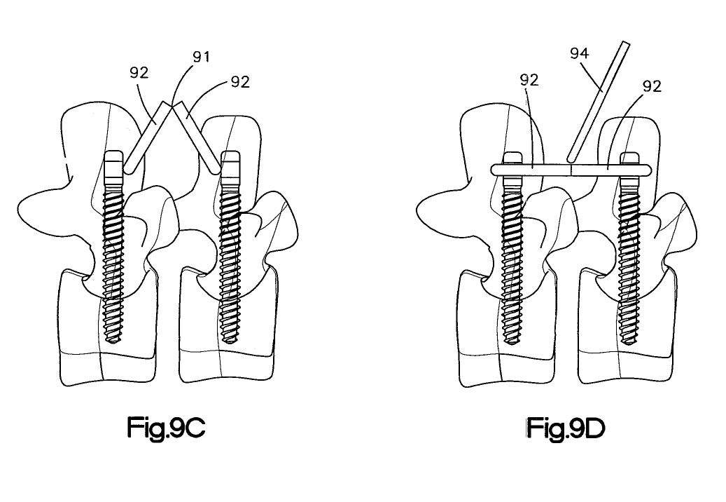

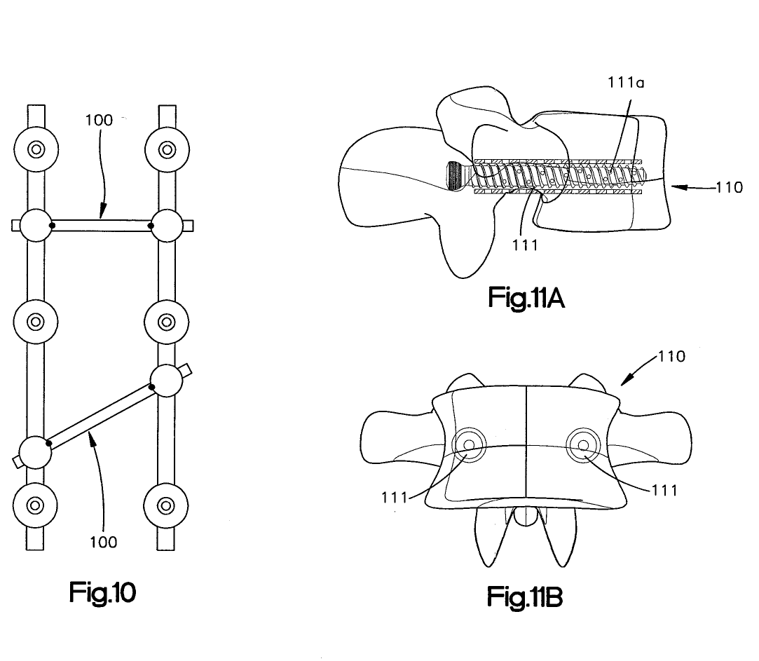

Referring to FIGS. 9A-9D, in spinal fusion, it is common to use pedicle screws and rods to stabilize a segment to fuse. However, the procedure is invasive and many efforts are taken to reduce invasiveness by use of mini-invasive pedicle screw insertion techniques. While it is possible to insert the pedicle screw through an almost stab incision, the rod is difficult to place because of the length and stiffness of the rod.

In a ninth preferred embodiment, an ultrasonically weldable rod 90 is flexible during insertion in an insertion configuration (FIGS. 9A and 9C) and stiff after insertion in an implanted configuration (FIGS. 9B and 9D). This can be achieved by composing the rod 90 of a string 91 and “pearls” 92, both constructed of PEEK, where the string 91 is connected to a needle 93 to allow insertion of the pearls 92 into the body, via a relatively minimally invasive technique. Once the string 91 and pearls 92 are in position, the string 91 is tightened to create contact between the pearls 92 and the pearls 92 are welded to each other by means of ultrasonic power, preferably an ultrasonic probe 94. Alternatively the pearls 92 can be welded to each other one by one upon insertion. In another alternative the rod 90 is composed of strings of PEEK and/or metal wires gathered in a bundle to form a rod. Since the wires/strings can slide freely along side each other the rod is flexible. After insertion or upon insertion the strings/wires are welded to each other by means of ultrasonic power or to prevent the sliding and alternatively turning of the bundle to transform the bundle into a stiff rod. The rod 90 may be composed of two pieces 92 connected by a hinge or portion of the string 91 and can be inserted into the body through an incision in a folded configuration (FIG. 9C). Once the two ends of the rod 90 have passed the skin level they can unfold until they contact the pedicle screw heads and until the rod 90 is substantially straight at its hinge, where it can be welded to make the hinge stiff. Alternatively, the rod 90 is composed of a flexible shaft (preferably hollow), a sheath and a device for injection of a self-curing polymer. The sheath covers the outside of the flexible shaft to prevent the polymer or cement from leaking through the shaft. Upon insertion the shaft is flexible and therefore can be introduced easily. After insertion of the shaft, the self-curing polymer or cement is injected into the inside of the flexible shaft where it hardens and causes the rod to become stiff. All of the above-described rods 90 could also deploy laser welding technique instead of ultrasonic welding or light curing systems instead of self-curing systems. In particular the PEEK-fiber bundle can be enhanced with glass fibers acting as light conductors for the laser welding as well as reinforcement to increase mechanical stiffness and strength.

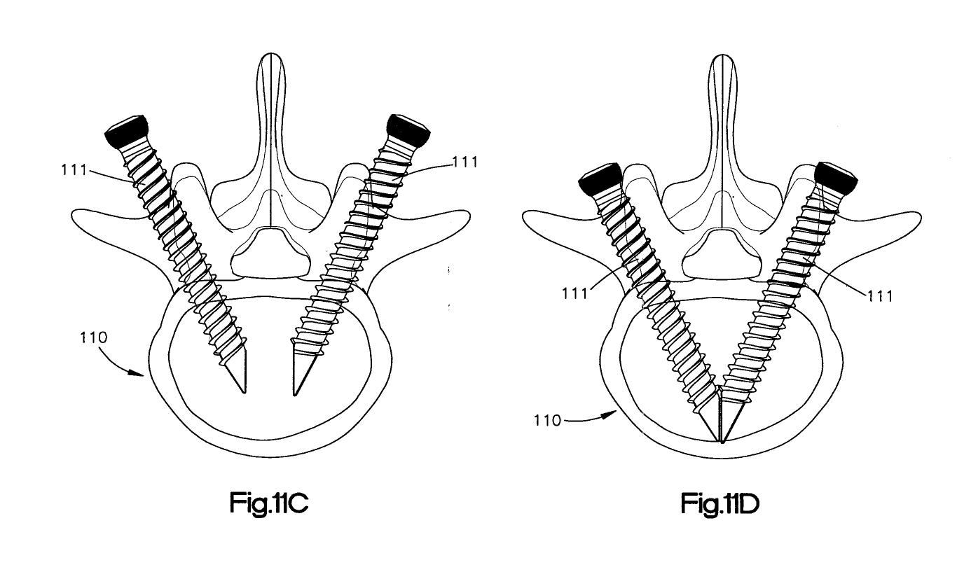

Referring to FIG. 10, in spinal fusion, pedicle screw/rod constructs can be reinforced by connecting left and right rods with cross connectors. However, placing of the connectors can be difficult and related implants are generally too bulky and, therefore, may disturb surrounding tissues.

In a tenth preferred embodiment, an ultrasonically weldable cross connector implant 100 has a low-profile/slim design. This is achieved more by deploying the ultrasonic welding technique because this allows for a slim interface between the cross connector 100 and a related rod. The rod and cross connector 100 are preferably constructed of PEEK, have PEEK coating or have adequate interfaces made of PEEK to allow for welding one to another. The rod and cross connector 100 are not limited to PEEK constructions and may be constructed of any thermoplastic material that is adaptable for ultrasonic welding or nearly any material that is biocompatible and may be welded, in situ.

Referring to FIGS. 11A-11D, in treatment of spinal disorders with pedicle screws, a common problem is loosening of the pedicle screw in the vertebral body. In such cases, if the support by a pedicle screw is still required, a revision is performed where the screw is replaced either by a screw of larger diameter or by the same screw in combination with cement. However, these options not optimal because of the limitations in pedicle diameter and poor handling of cement.

In a eleventh preferred embodiment, an ultrasonically weldable pedicle screw anchor 110 is comprised of a dowel 111 made of a polymer or thermoplastic, such as PEEK, that can be inserted in an existing hole in the vertebral body. The dowel 111 can then be expanded by inserting a pedicle screw or a similar but nonthreaded device into the dowel 111 and fixing the pedicle screw to the dowel 111 by means of ultrasonic power. Alternatively the dowel 111 can be preloaded with cement, the cement being extruded through appropriate holes 111a in the dowel 111 and into the vertebral body surrounding the dowel 111 thus improving load transfer to the bone. An alternative solution is to provide pedicle screws that are at least partially made of PEEK or another thermoplastic material, with the PEEK being positioned at least at the tip in order to weld the left and right pedicle screw to each other at their tips inside the vertebral body.

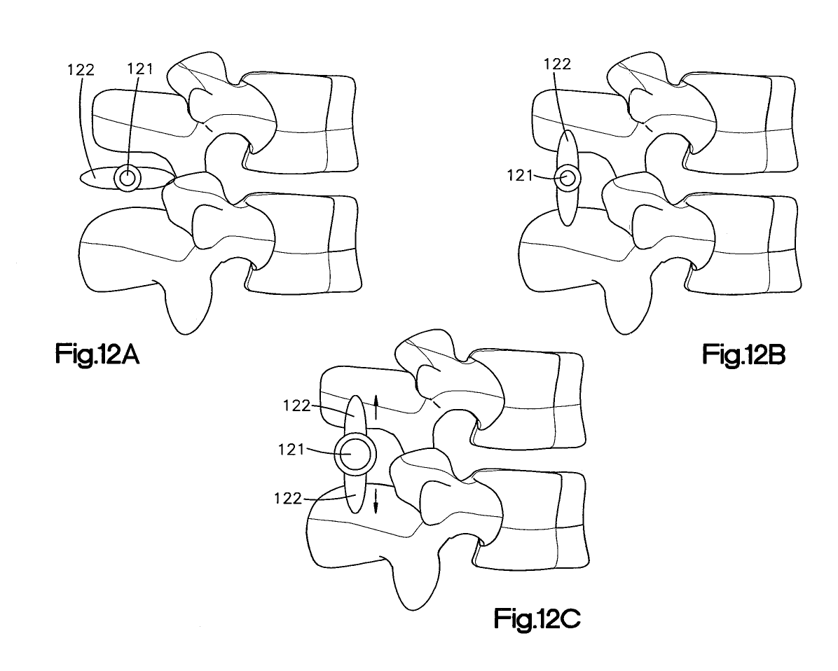

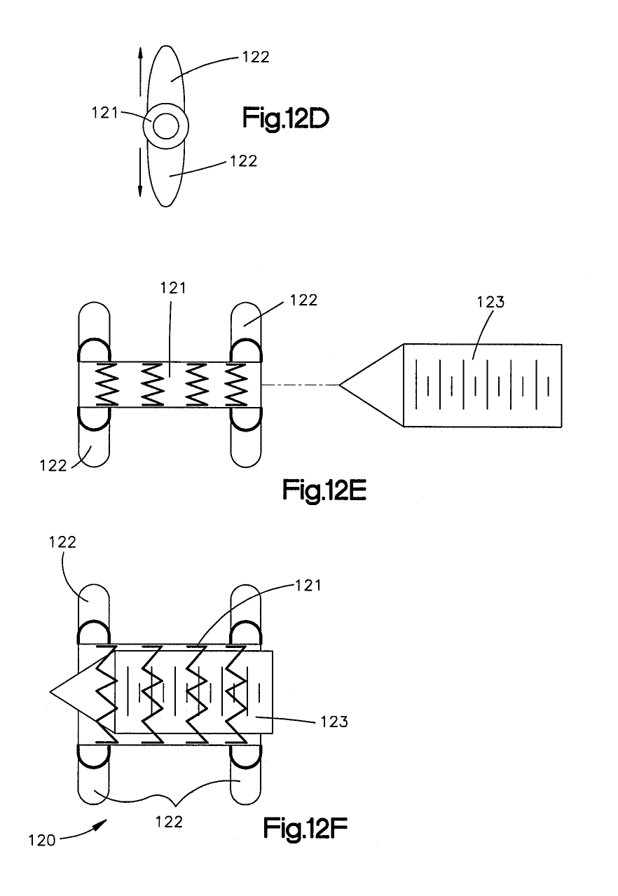

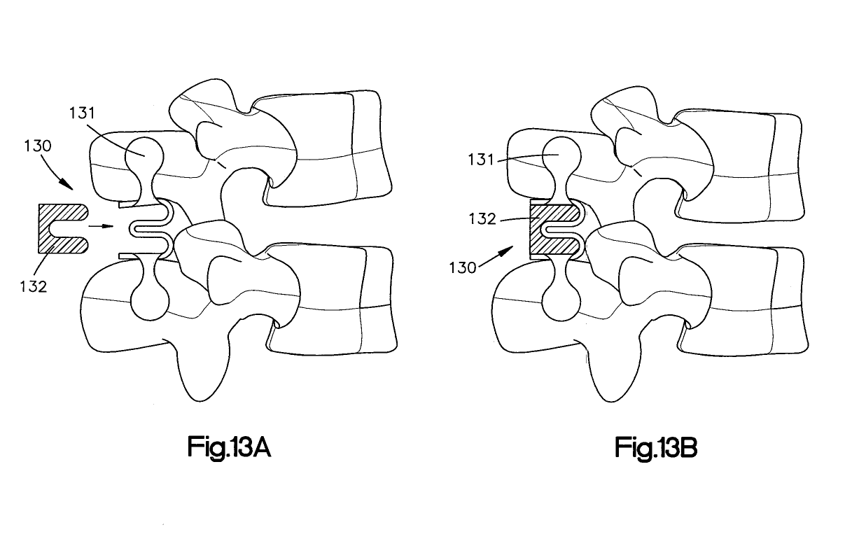

Referring to FIGS. 12A and 12F, in treatment of dynamic spinal stenosis, interspinous spacers are used to distract adjacent spinous processes to relieve neural structures from pressure. In this type of treatment it is particularly desirable to perform implantation in a percutaneous approach thus causing little or no damage on tissues.

In a twelfth preferred embodiment, an ultrasonically weldable expandable interspinous spacer 120 is composed of a cylindrical body 121 and two pairs of wings 122 extending from the body 121 substantially perpendicular to a longitudinal axis of the body 121. The body 121 has a hole parallel to the longitudinal axis and a series of cuts (e.g. z-shaped) to allow for expansion (similar to dowels). The spacer 120 is being inserted between adjacent spinous processes with the wings 122 pointing in anterior-posterior direction and subsequently, the wings 122 are rotated by ninety degrees (90°) along the longitudinal axis in order to position the wings 122 on either side of the spinous processes. A screw or plug 123 is preferably inserted into the body 121 of the spacer 120, thus causing the body 121 to expand radially and distract the spinous processes. The plug 123 is then welded to the body 121 of the spacer 120 in order to prevent the construct from disassembly. Alternatively the plug/screw 123 can be of conical shape in order to allow for continuous distraction by the desired amount.

Referring to FIGS. 13A and 13B, for the treatment of spinal stenosis, interspinous spacers are widely used to distract the spinous processes and keep them distracted and to increase stability of the decompressed spinal segment. However, depending on several factors as patient age, activity, segmental mobility etc., such a device should offer concurring behavior. This mainly concerns implant stiffness: for a younger, active patient, a softer implant is more suitable, while for a patient who requires an increase in stability, a stiffer implant is preferred. Therefore, it is desirable to have an implant allowing adjustment of the stiffness.

In a thirteenth preferred embodiment, an ultrasonically weldable interspinous process blocking implant 130 includes a flexing portion 131 with a relatively low stiffness a number of plugs 132 of different stiffness that can be introduced into the flexing portion 131 to increase the spacer stiffness to a desired amount. The plug 132 may be shaped to fit into the inside of the W-shaped flexing portion 131. After intraoperative insertion of the plug 132 it is then attached to the flexing portion 131, preferably by means of ultrasonic power.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

We claim:

1. A spinal implant for augmenting or supporting a patient's spine including vertebrae and intervertebral discs, the spinal implant comprising:
a first component constructed of a thermoplastic material;
a second component constructed of the thermoplastic material, the first and second components configured for implantation into the spine such that the first component contacts the second component at a welding point in an initial implanted position; and
an ultrasonic probe including a tip, the tip configured to selectively contact at least one of the first and second components in the initial implanted position to transform the welding point to a weld joint in a final implanted position, the first component being fixed to the second component at the weld joint in the final implanted position.
